(19) United States
(12) Patent Application Publication
(10) Pub. No.: US 2023/0405042 A1
(43) Pub. Date: Dec. 21, 2023

(54) Compositions to Modify Intestinal Nutrient Absorption, Methods of Making and Uses Thereof

(71) Applicant: McMaster University, Hamilton (CA)

(72) Inventors: Jonathan Schertzer, Hamilton (CA); Fernando Forato Anhe, Hamilton (CA); Han Fang, Hamilton (CA)

(21) Appl. No.: 18/198,154

(22) Filed: May 16, 2023

Related U.S. Application Data
(60) Provisional application No. 63/342,292, filed on May 16, 2022.

(51) Int. Cl. A61K 31/765; A61K 38/44; A61K 38/26; A61K 31/155; A61K 35/741; A61P 3/10

(52) U.S. Cl. CPC A61K 31/765; A61K 38/443; A61P 3/10; A61K 31/155; A61K 35/741; A61K 38/26

(57) ABSTRACT

The present application relates to methods of lowering at least one of blood glucose, liver fat, liver glycogen, insulin or insulin resistance in a mammal, for example, mammals who are obese, or have metabolic disease, fatty liver disease, pre-diabetes or type 2 diabetes. In an embodiment, a method comprises administering to a mammal a polymer comprising L-lactate monomer, or a functionally equivalent derivative or fragment thereof, which inhibits or at least reduces D-lactate transport across the intestinal barrier. Compositions useful in the methods are also provided.

### FIELD

The present application relates to the fields of obesity and/or metabolic disease, and in particular, to compositions which lower body weight, lower liver fat, lower liver glycogen and/or lower blood glucose, and/or improve blood glucose, insulin control and/or insulin sensitivity, in an individual, methods of making and uses thereof.

### BACKGROUND

Obesity is characterized by expanded adipose tissue, ectopic fat deposition (e.g., fatty liver), higher blood insulin, insulin resistance and higher blood glucose that is poorly controlled in the normal blood glucose range. Obesity promotes type 2 diabetes (T2D) and non-alcoholic fatty liver disease (NAFLD), which are interlinked chronic diseases that have a high health and economic burden. These metabolic disturbances are also interlinked and as an example chronically higher blood insulin contributes to increased obesity. There are currently no approved therapies for NAFLD, which can lead to liver cirrhosis and cancer. Diets that restrict certain macronutrients and lower caloric intake can mitigate obesity and promote remission of T2D and potentially NAFLD. However, long term diet adherence is a major problem. Bariatric surgery lowers intestinal nutrient absorption and is the most efficacious treatment for T2D. However, surgery is expensive and not accessible to the population at a level needed to combat T2D or NAFLD.

Therefore, there is a need to discover modifiers of intestinal nutrient absorption that offer better compliance and accessibility than current diet and surgical treatments.

Bacteria-derived molecules influence host metabolism, body weight and particularly adipose tissue and liver metabolism partly through the portal circulation and hepatobiliary system, thereby regulating the development of fatty liver disease and insulin resistance. Fatty liver disease and T2D are associated with perturbations in the intestinal microbiome, including alterations in taxonomy (i.e., type of bacteria), predicted function of bacteria and bacterial metabolites. It is critical to define molecules derived from bacteria that regulate the fundamental processes that control whether fat is stored in the liver and whether carbohydrates (i.e., glucose) are released into the blood by the liver.

The Con cycle describes that L-lactate derived from skeletal muscle glycolysis fuels the liver to produce glucose. However, while providing extra L-lactate by injection can reverse low blood glucose, there is evidence that injection of L-lactate does not always raise blood glucose above normoglycemic levels. In addition, Cori & Cori found that oral delivery or injection of D-lactate in rodents leads to glycogen deposition in the liver, whereas L-lactate delivery “hardly forms any liver glycogen”. While D-lactate and L-lactate are absorbed at the same rate by the intestine, people with diabetes and rodent models of diabetes both have higher levels of blood and urine D-lactate.

“Gut substrate trap” therapies do not depend on diet adherence and have an advantage over bariatric surgery because they can be selective for specific nutrients or metabolites. Obesity, T2D and NAFLD are associated with changes in the intestinal microbiome, including alterations in bacterial taxonomy, function and bacterial metabolites and the gut microbiota can regulate blood glucose by altering hepatic gluconeogenesis.

### SUMMARY

The present invention provides methods and compositions to lower body weight, liver fat, liver glycogen, and/or blood glucose, and/or improve blood glucose, blood insulin control and insulin sensitivity in an individual. The method is based on the findings that D-lactate in the host blood is produced primarily by gut bacteria, gut bacteria-produced D-lactate is absorbed by the intestine, enters the blood circulation and microbial-derived D-lactate is a substrate that forms host liver glycogen, liver fat and blood glucose. These findings demonstrate that lowering intestinal absorption of D-lactate from the gut lumen also lowers D-lactate in the blood, which then lowers liver fat, liver glycogen and blood glucose.

Thus, in one aspect of the invention, a method of lowering at least one of blood glucose, liver fat and liver glycogen is provided comprising administering to a mammal a polymer comprising L-lactate monomer, or a functionally equivalent derivative or fragment thereof, which inhibits or at least reduces D-lactate transport across the intestinal barrier.

In another aspect, a method of lowering insulin and/or insulin resistance in a mammal is provided, comprising administering to a mammal a polymer comprising L-lactate monomer, or a functionally equivalent derivative or fragment thereof, which inhibits or at least reduces D-lactate transport across the intestinal barrier.

In another aspect, a method of lowering at least one of blood glucose, liver fat, liver glycogen, blood insulin and insulin resistance is provided comprising the step of inhibiting or reducing D-lactate transport across the intestinal barrier.

In a further aspect of the invention, a composition is provided comprising a polymer comprising L-lactate monomer in combination with one or more agents that promote degradation or metabolism of D-lactate.

In another aspect, a composition comprising a polymer comprising L-lactate monomer linked or bound to non-biodegradable or non-digestible agent in combination with a pharmaceutically acceptable carrier is provided.

Other features and advantages of the present application will become apparent from the following detailed description. It should be understood, however, that the detailed description and the specific examples, while indicating embodiments of the application, are given by way of illustration only and the scope of the claims should not be limited by these embodiments but should be given the broadest interpretation consistent with the description as a whole.

### DRAWINGS

The embodiments of the application will now be described in greater detail with reference to the attached drawings in which:

FIG. 1 shows the gut microbiota is the main source of circulating D-lactate, but not L-lactate, in the host in an exemplary embodiment of the disclosure. (a) Germ-free mice were obtained from an axenic gnotobiotic unit and conventionalized mice were mice that were born under germ-free conditions but exposed to the bedding from specific pathogen free (SPF) mice (i.e., colonized) for 23 weeks. Blood samples were collected from mice bred in germ-free or conventionalized conditions. Plasma L-lactate (b, e) and D-lactate (c, f), as well as their ratio (d, g), were determined using a fluorometric assay both after 24 h fasting (b-d) and in random-fed mice (e-g). The number of independent biological replicates tested were: (b-d) Germ-free n=22 and Conventionalized n=12; (e-g) Germ-free n=19 and Conventionalized n=12. P values were calculated using Mann-Whitney U tests and considered statistically significant at P<0.05. The graphs depict the mean±SEM, where each dot is a separate mouse.

FIG. 2 shows gut microbiota regulate liver glycogen and triglycerides in an exemplary embodiment of the disclosure. (a) Livers were collected from age-matched mice bred in germ-free or SPF conditions. Liver glycogen (b, d) and triglycerides (c, e) were determined using a colorimetric assay both after 12 h fasting (b, c) and 12 h-fasting then re-feeding standard chow diet for 2 h (d, e). The number of independent biological replicates tested were: (b, c) Germ-free n=17-19 and SPF n=6; (d, e) Germ-free n=21 and SPF n=7. P values were calculated using Mann-Whitney U tests and considered statistically significant at P<0.05. The graphs depict the mean SEM, where each dot is a separate mouse.

FIG. 3 shows D-lactate fuels blood glucose and liver glycogen and triglycerides in an exemplary embodiment of disclosure. (a) Liver and blood samples were collected from mice receiving either 4 g/kg of L-lactate or 4 g/kg D-lactate via i.p. injection. Liver glycogen (b, d) and triglycerides (c, e) were determined using a colorimetric assay both after 12 h fasting (b, c) and 12 h-fasting re-fed standard chow diet for 2 h (d, e). Lactate tolerance tests (f) were performed in mice after 12 h fasting. Blood glucose was measured using a MediSure® glucometer at 0, 20, 60, 80, 100, 120, 150, 180, 210 min after i.p. injection of 4 g/kg of L-lactate or 4 g/kg D-lactate. Area under the curve (AUC) (g) was calculated. The number of independent biological replicates tested were: (b, c) L-lactate n=8 and D-lactate n=8; (d, e) L-lactate n=9 and D-lactate n=8; (f, g) L-lactate n=11 and D-lactate n=9. P values were calculated using Mann-Whitney U tests (b-e, g) and Two-way repeated measures ANOVA with Bonferroni's multiple comparison test (f). P values were considered statistically significant at P<0.05. The graphs depict the mean±SEM, where each dot is a separate mouse.

FIG. 4 shows that colonization of mice with bacteria that produces higher microbial D-lactate increases blood glucose in an exemplary embodiment of the disclosure. (a) Germ-free mice were monocolonized with Lactobacillus intestinalis ASF360 (D-lactate^{HIGH}, produce high levels of D-lactate) or Lactobacillus reuteri 149 (D-lactate^{LOW}, produce low levels of D-lactate) at week 0 and then re-colonized at week 6. Blood and fecal samples were collected throughout the study. D- and L-lactate production (b) was determined in bacterial culture media of both D-lactate^{HIGH} and D-lactate^{LOW} strains. Body weight (c), food intake (d), changes in random-fed blood glucose (e) were monitored weekly. Fasting blood glucose (f) were determined using a MediSure® glucometer at week 2 and 7 after 6-12 h fasting. Serum and fecal L-lactate (g, h) and D-lactate (i, j) were determined using a fluorometric assay at week 2 and 7. The number of independent biological replicates tested were: (b) D-lactate^{HIGH}=3 and D-lactate^{LOW}=3; (c-e) D-lactate^{HIGH}=10-16 and D-lactate^{LOW}=14; (f) D-lactate^{HIGH}=9-11 and D-lactate^{LOW}=13; (g-j) D-lactate^{HIGH}=11-15 and D-lactate^{LOW}=13. P values were calculated using unpaired t test (b), Two-way repeated measures ANOVA with Bonferroni's multiple comparison test (c-e) and Mann-Whitney U tests (f-j). P values were considered statistically significant at P<0.05. The graphs depict the mean±SEM, where each dot is a separate mouse.

FIG. 5 shows that a novel substrate trap that captures microbial D-lactate lowers blood glucose and liver fat in an exemplary embodiment of the disclosure. (a) PDL20 (Poly-DL-Lactide) is predicted to capture L-lactate and D-lactate, PD24 (Poly-D-Lactide) is predicted to capture L-lactate, and PL24 (Poly-L-Lactide) is predicted to capture D-lactate as depicted. Changes in blood glucose (b) were measured 4 h after a single oral administration of PDL20, PD24 or PL24 to lean chow-fed mice upon fasting. In a separate cohort, 35-week-old mice were on a 60% high fat diet (HFD) for 29 weeks and then switched to 60% HFD+10% PL24 for 1 day. Liver triglycerides (c) were determined using a colorimetric assay in these obese mice after 18 h fasting and subsequently re-feeding for 2 h with 60% HFD or 60% HFD+10% PL24. The number of independent biological replicates tested were: (b) PDL20 n=7, PD24 n=9 and PL24 n=9; (c) CTL n=11 and PL24 n=11. P values were calculated using One-way ANOVA with Tukey's multiple comparison test (b) and Mann-Whitney U tests (c). P values were considered statistically significant at P<0.05. The graphs depict the mean±SEM, where each dot is a separate mouse.

FIG. 6 shows that treatment with the D-lactate trap PL24 lowers blood glucose, insulin, and insulin resistance in diet-induced obese mice in an exemplary embodiment of the disclosure. (a) Twenty-three-week-old mice were on a 60% HFD for 17 weeks, then half of the mice were switched to 60% HFD+10% PL24 diet, the other half were remained on 60% HFD for 7 weeks. Body weight (b) and (e) cumulative food intake (c) were monitored weekly throughout the study. Stool samples were collected overnight (12 h) at week 4 for assessment of L-lactate (d) and D-lactate (g) fecal output. Plasma L-lactate (e, f) and D-lactate (h, i) were measured upon 4 h and 12 h fasting at week 4 and in random-fed mice at week 4 and week 6. At week 7 of treatment, blood glucose (j) and insulin (k) were monitored during fasting, and insulin resistance was estimated by the HOMA-IR index (l). The number of independent biological replicates tested were: (b, e, f, h-l) CTL n=13 and PL24=8; (d, g) CTL n=13 and PL24 n=7. Food intake was measured in each cage containing multiple mice (c) CTL n=3 and PL24 n=3. P values were calculated using Two-way repeated measures ANOVA with Bonferroni's multiple comparison test (b, c) and Mann-Whitney U tests (d-l). P values were considered statistically significant at P<0.05. The graphs depict the mean±SEM, where each dot is a separate mouse.

FIG. 7 shows poly-L-lactide lowers blood glucose, insulin and insulin resistance in a polymer length dependent manner in diet-induced obese mice in an exemplary embodiment of the disclosure. The affect of polymer length is shown on a) body weight and b) food intake, as well as on c) serum L-lactate levels, d) serum D-lactate levels, e) fasting blood glucose, f) fasting insulin and g) HOMA-IR index.

### DETAILED DESCRIPTION

A method of lowering at least one of blood glucose, liver fat and liver glycogen in a mammal is provided comprising administering to the mammal a polymer comprising L-lactate monomer, or a functionally equivalent derivative or fragment thereof, which inhibits or at least reduces D-lactate transport across the intestinal barrier. The present method comprises administration of a polymer comprising L-lactate monomer which inhibits or reduces D-lactate transport across the intestinal barrier, or a functionally equivalent derivative or fragment thereof. The term “functionally equivalent” as used herein refers to derivatives or fragments of the L-lactate-containing polymer which retain the function of L-lactate-comprising polymer to inhibit or reduce D-lactate transport across the intestinal barrier.

The phrase “inhibits or reduces D-lactate transport across the intestinal barrier” refers to partial or total inhibition of D-lactate transport across the intestinal barrier and may result from trapping D-lactate within the intestinal lumen or at the mucosal surface, and/or metabolizing D-lactate within the intestinal lumen into compounds that do not adversely influence blood glucose, liver glycogen and fat in a mammal. Trapped D-lactate within the intestinal lumen or mucosal surface is then forced to be excreted in feces, thereby preventing or minimizing D-lactate as a substrate to form liver fat and blood glucose irrespective of other dietary changes. Sources of D-lactate within the intestine include ingested materials (foods), host-derived D-lactate and D-lactate produced by microbiota.

The “intestinal barrier” or “intestinal mucosal barrier” is a heterogeneous entity composed of physical, biochemical and immune elements. The physical component is the intestinal epithelial layer which separates the intestinal lumen from the body. The intestinal epithelial layer is coated with a layer or layers of mucus that protects the epithelial layer from harmful digestive enzymes and microorganisms, and prevents passage of large particles into the body. The epithelial layer comprises junctional complexes that mediate passage of ions, nutrients and other substances from the lumen into the body. Gut microbiota also influence barrier functions of the epithelial layer. Biochemical elements include bile, gastric acid, defensins, lysozyme and leptin, and immunological elements include antimicrobial peptides, secretory immunoglobulin A, and a variety of immune cells such as dendritic cells, macrophages, lymphocytes and T cells.

A polymer comprising L-lactate monomer refers to polylactic acid or polylactide comprising L-lactate monomers, including poly-L-lactic acid, poly-D,L-lactic acid, poly-L-lactide and poly-DL-lactide. As one of skill in the art will appreciate, polylactic acid (PLA) may be prepared from lactate monomer or lactide monomer (via a ring opening). In one embodiment, the L-lactate-comprising polymer comprises at least about 15 lactide monomer units (PL15 or PDL15), preferably at least about 20 lactide monomer units (PL20 or PDL20), such as 21, 22, 23, 24, 25, 30, 35, 40, 50, or more lactide monomer units. In another embodiment, the polymer comprises less than 200 lactide monomer units (PL200 or PDL200), preferably less than 100 lactide monomer units (PL100 or PDL100). In another embodiment, the polymer comprises 20-65 lactide monomer units. In a further embodiment, the polymer comprises a poly-L-lactide polymer. In other embodiments, the polymer comprises a poly-D,L-lactide polymer comprising L-lactide and D,L-lactide monomers or only D,L-lactide monomer. The ratio of L-lactide to D-lactide, or L-lactate to D-lactate, in a poly-D,L-lactide polymer or poly-D,L-lactic acid, may vary. Generally, the higher the amount of L-lactate monomer in a poly-D,L-lactide polymer, the more efficacious the poly-D,L-lactide. In one embodiment, the poly-D,L-lactide polymer for use has a 50:50 ratio of L-lactate and D-lactate monomer. Alternatively, the L-lactate-comprising polymer comprises a corresponding number of lactate monomer, e.g. two lactate monomers for every lactide monomer.

A derivatized or modified L-lactate-comprising polymer may also be used in the present method. For example, in one embodiment, the L-lactate-comprising polymer is linked or bound to non-biodegradable or non-digestible microparticles that promote a higher dwell time in the intestinal lumen and which may additionally buffer pH changes to the polymer. Examples of non-biodegradable or non-digestible microparticles include but are not limited to, dietary fiber particles such as beta-glucan soluble fiber, psyllium husk, cellulose, guar gum, pectin, mucilage, locust bean gum, hydroxypropylmethylcellulose, arabinoxylan, alginate, inulin and inulin-type fructans, high amylose starch (resistant starch 2), galactooligosaccharide, polydextrose, resistant maltodextrin/dextrin, cross linked phosphorylated resistant starches 4 (RS4), glucomannan, acacia (gum arabic) and plant cell wall fibers (a broad category that includes fibers like sugar cane fiber, apple fiber, among others). L-lactate-comprising polymer may also be bound to a delivery vehicle, such as a polymer, which provides stability to the L-lactate-comprising polymer by minimizing acid-catalyzed hydrolysis and/or enzymatic degradation. Exemplary polymers for use as a delivery vehicle include polyethylene glycol.

A fragment of an L-lactate-comprising polymer may also be used in the present method. For example, a fragment of poly-L-lactide or poly-DL-lactide consisting of at least about 15, and preferably at least about 20 lactide monomer units is useful in the present method. In embodiments, fragments up to and including 100 lactide monomer units are used in the present method, such as fragments having 24-65 coupled lactide monomers. As one of skill in the art will appreciate, the use of a mixture of poly-lactide fragments of various lengths may also be used.

The L-lactate-comprising polymer, or a functionally equivalent derivative or fragment thereof, which may collectively be referred to herein as “L-lactate-comprising polymer”, is administered to the mammal to lower at least one of blood glucose, liver fat, liver glycogen, insulin and/or insulin resistance. Thus, the present L-lactate-comprising polymers are useful to treat metabolic disease, including obesity. In embodiments, L-lactate-comprising polymer, or a functionally equivalent derivative or fragment thereof, is useful to treat a mammal with non-alcoholic fatty liver disease (NAFLD), pre-diabetes, or type 2 diabetes (T2D).

The present L-lactate-comprising polymers may be administered to a mammal in need of treatment using any suitable mode of administration, including but not limited to, oral, sublingual, rectal, topical, by inhalation, or via parenteral administration such as intravenous, intramuscular or subcutaneous administration.

For use to inhibit or reduce D-lactate transport across the intestinal barrier, a therapeutically effective amount of L-lactate-comprising polymer, or a functionally equivalent derivative or fragment thereof is administered to a mammal. As used herein, the term “mammal” is meant to encompass, without limitation, humans, domestic animals such as dogs, cats, rodents and like, and ruminant mammals such as horses, cattle, swine, sheep, goats and the like, as well as non-domesticated animals. The term “therapeutically effective amount” is an amount of the L-lactate-comprising polymer, or a functionally equivalent derivative or fragment thereof required to inhibit or at least reduce D-lactate transport across the intestinal barrier so as to lower at least one of blood glucose, liver fat, liver glycogen, insulin and/or insulin resistance, while not exceeding an amount which may cause significant adverse effects. Suitable therapeutically effective dosages will vary on many factors including the nature of the condition to be treated as well as the particular individual being treated. In an embodiment a daily dosage of L-lactate-comprising polymer, or a functionally equivalent derivative or fragment thereof is in a range of about 0.02 g/kg to 20 g/kg.

The L-lactate-comprising polymer, or a functionally equivalent derivative or fragment thereof, may be administered in a total daily effective amount to a mammal in need thereof, once a day, or 2 or more times per day, and for a period ranging from one day to chronic or long-term administration. Thus, the daily effective amount may be divided into 2, 3, 4, 5, 6 or more portions to be administered throughout the day over 1 or more days or over 1 or more weeks, i.e. 2, 3, 4, 5, 6, 7, 8, 9, 10 or more days or weeks.

In another embodiment, the L-lactate-comprising polymer, or a functionally equivalent derivative or fragment thereof, may be formulated for controlled, sustained or extended release. For example, the L-lactate-comprising polymer may be contained a diffusion or dissolution system in which release of the L-lactate-comprising polymer is sustained or extended over a period of time and correlates with the dissolution of one or more materials within a coating or matrix that contains the L-lactate-comprising polymer to release the L-lactate-comprising polymer. Such formulations permit steady or pulsed release of L L-lactate-comprising polymer, and/or may be adapted to contain more than 1 daily dosage to be delivered over 2 or more days.

The L-lactate-comprising polymer, or a functionally equivalent derivative or fragment thereof, may be administered either alone or in combination with at least one pharmaceutically acceptable adjuvant to form a composition, for use in treatments in accordance with embodiments of the invention. The expression “pharmaceutically acceptable” means acceptable for use in the pharmaceutical and veterinary arts, i.e. not being unacceptably toxic or otherwise unsuitable. Examples of pharmaceutically acceptable adjuvants include diluents, excipients and the like. Reference may be made to “Remington's: The Science and Practice of Pharmacy”, 21st Ed., Lippincott Williams & Wilkins, 2005, for guidance on drug formulations generally.

The selection of adjuvant depends on the intended mode of administration of the polymer or composition thereof. In one embodiment of the invention, the polymer is formulated for administration by infusion, or by injection either subcutaneously or intravenously, and are accordingly utilized as aqueous solutions in sterile and pyrogen-free form and optionally buffered or made isotonic. Thus, the compounds may be administered in distilled water or, more desirably, in saline, phosphate-buffered saline or 5% dextrose solution. Compositions for oral administration via tablet, capsule or suspension are prepared using adjuvants including sugars, such as lactose, glucose and sucrose; starches such as corn starch and potato starch; cellulose and derivatives thereof, including sodium carboxymethylcellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil and corn oil; polyols such as propylene glycol, glycerine, sorbital, mannitol and polyethylene glycol; agar; alginic acids; water; isotonic saline and phosphate buffer solutions. Wetting agents, lubricants such as sodium lauryl sulfate, stabilizers, tableting agents, anti-oxidants, preservatives, colouring agents and flavouring agents may also be present. Formulations for administration intranasally, or by inhalation, may also be prepared in saline or other suitable buffer and/or propellant adjuvants, to be nebulized to form a liquid aerosol for inhalation by mouth or nasally. Other adjuvants may also be added to the composition regardless of how it is to be administered, for example, anti-microbial agents may be added to the composition to prevent microbial growth over prolonged storage periods.

Alternatively, the present polymer or functionally equivalent derivative or fragment thereof may be combined within food or combined within a beverage for oral consumption one or more times per day.

L-lactate-comprising polymer, or functionally equivalent derivatives or fragments thereof may be administered together with (either in combination, or individually, simultaneously or sequentially) one or more agents that promote degradation or metabolism of D-lactate to facilitate inhibition of D-lactate transport across the intestinal barrier. Examples of agents suitable for this purpose include but are not limited to enzymes, such as D-lactate dehydrogenase, D-lactate dehydrogenase (cytochrome), D-lactate dehydrogenase (cytochrome c-553) and D-lactate dehydratase (glyoxalase 3); bacteria, such as bacteria which express D-lactate-metabolizing enzymes such as Lactobacillus bulgaricus, Escherichia coli, Fusobacterium nucleatum, Pseudomonas aeruginosa, plant Arabidopsis thaliana, yeast Hansenula polymorpha, yeast Saccharomyces cerevisiae and Desulfovibrio vulgaris; and agents which catalyze or facilitate D-lactate degradation or metabolism such as ferricytochrome c and/or ferricytochrome c-.

In another embodiment, L-lactate-comprising polymer, or functionally equivalent derivatives or fragments thereof may be administered together with a therapeutic agent such as a therapeutic agent useful to treat obesity, type 2 diabetes or fatty liver, including but not limited to, GLP-1-based drugs, GLP-1/GIP co-agonists and GLP-1/GIP/glucagon tri-agonists agonists, SGLT2 inhibitors, metformin and statins.

L-lactate-comprising polymer, or functionally equivalent derivatives or fragments thereof may be administered encapsulated within hydrogels (macro- or micro-gels) such as poly (lactic-co-glycolic acid), poly (ε-caprolactone) (PCL), poly (3-hydroxybutyrate-co-3-hydroxyvalerate) (PHBV), chitosan, cellulose and poly (acrylates) including poly (methyl methacrylate) (PMMA) and electrospun fibres prepared from biocompatible polymers, such as those that may mimic conditions similar to those occurring in the natural extracellular matrix (ECM), which provide a product presenting high specific surface area to enhance adsorption. Buffers such as calcium carbonate and sodium bicarbonate may additionally be included in the hydrogel/microgel to buffer pH. In one embodiment, the hydrogel or microgel is complexed with poly(ethylene glycol) (e.g. PEGylated) to provide stability against hydrogel or microgel degradation, such as acid-catalyzed hydrolysis (e.g. of ester groups) and enzymatic degradation.

### Definitions

Unless otherwise indicated, the definitions and embodiments described in this and other sections are intended to be applicable to all embodiments and aspects of the present application herein described for which they are suitable as would be understood by a person skilled in the art.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±10% of the modified term if this deviation would not negate the meaning of the word it modifies.

As used in this application, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

Embodiments of the invention are described by reference to the following detailed Examples which are not to be construed as limiting.

### EXAMPLES

### Example 1. Materials and Methods

Animals: All procedures were approved by the McMaster University Animal Ethics Board (animal utilization protocol #: 16-02-96). Specific pathogen free (SPF) C57BL/6N mice were purchased from Taconic or sourced from in-house colonies and kept on a standard chow diet (Teklad 22/5 diet, catalog #8640). SPF C57BL/6N diet-induced obese (DIO)-mice were purchased from Taconic and kept on high-fat diet with 60% of energy derived from saturated fat (HFD, Research Diets, D12492). Germ-free C57BL/6N mice were obtained from in-house colonies bred in the Famcombe Gnotobiotic Unit at the McMaster Animal Facilities. Conventionalized mice were obtained by mixing the dirty bedding from SPF mice to that of germ-free mice weekly for 23 weeks.

Biochemical analysis: Plasma insulin was assessed using high sensitivity mouse insulin ELISA kit (Toronto Bioscience, Cat #32270). L-lactate and D-lactate were determined using a PicoProbe™ L-lactate Fluorometric Kit (BioVision, Catalog #K638) and a PicoProbe™ D-lactate Fluorometric Kit (BioVision, Catalog #K668), respectively. For D-lactate quantification, plasma samples were diluted 1:10-1:15 in deionized water; for L-lactate, plasma samples were diluted 1:150. The diluted plasma samples (5-8 μL) were assayed. Fecal samples were resuspended in deionized water (1:50 [w:v]) and vortexed at 2000 rpm, 37° C. for 15 min. After a short spin to bring all solid particles to the bottom, the fecal slurries were diluted 1:300 in deionized water and 5 μL were assayed for both D- and L-lactate measurement.

Polylactide polymers: Poly-DL-Lactide (PDL20, Purasorb®PDL), poly-D-Lactide (PD24, Purasorb®PD) and poly-L-Lactide (PL24, Purasorb®PL) were purchased from Corbion Purac (Gorinchem, Netherlands). The polymers were reduced to a fine powder using a POLYMIX PX-MFC 90 D (Kinematica, Inc., New York, USA) and stored at −20° C.

Polylactide acute administration: Powdered polymers were mixed with saline (1:1 [w:v]) to yield a uniform resuspension. Chow-fed mice were 24 h fasted and subsequently fed with small increments of saline-sorbed PDL20, PD24 or PL24 (approximate final dose=100 mg/mouse). Control mice were gavaged with saline. Blood glucose was monitored before (0 h) and 4 h after administration of different polymers.

Glucose tolerance tests: Mice were 6 h fasted (8:00 AM-2:00 PM) and tail blood glucose monitoring was performed before (0 min) and 20, 30, 40, 60, 90 and 120 min after oral glucose (4 g/kg) using a MediSure® glucometer.

Long-term feeding with PL24: DIO-mice were kept on HFD for 17 weeks. Mice were thereafter switched to a HFD supplemented with 10% PL24 (w:w) whereas a control group was kept on regular HFD for 7 additional weeks. Body weight, food intake and random-fed blood glucose was monitored weekly. Body fat composition was measured after 4 weeks of dietary intervention with PL24 using whole body MRI (Bruker Minispec LF90-II). At week 4, mice were individually placed in clean cages without bedding and feces were collected after 12 h for lactate output assessment. At weeks 4 and 6, blood was drawn by tail sampling from mice on a random-fed state and used for insulin and lactate quantification. At week 7, blood glucose, insulin and lactate were monitored in mice after 4 h and 12 h fasting. HOMA-IR was used to assess fasting insulin resistance and was calculated as follows: (Fasting Insulin [mIU/L]×Fasting Glucose [mmol/L])/22.5).

Statistical analysis: Mann-Whitney U-test was used to compare two groups. One-way analysis of variance (ANOVA) followed by Tukey's multiple comparison test was used to compare more than two groups. Two-way repeated measures ANOVA with Bonferroni's post hoc test was applied to compare between groups throughout several time-points. Statistical significance was accepted at p<0.05.

### Results and Discussion

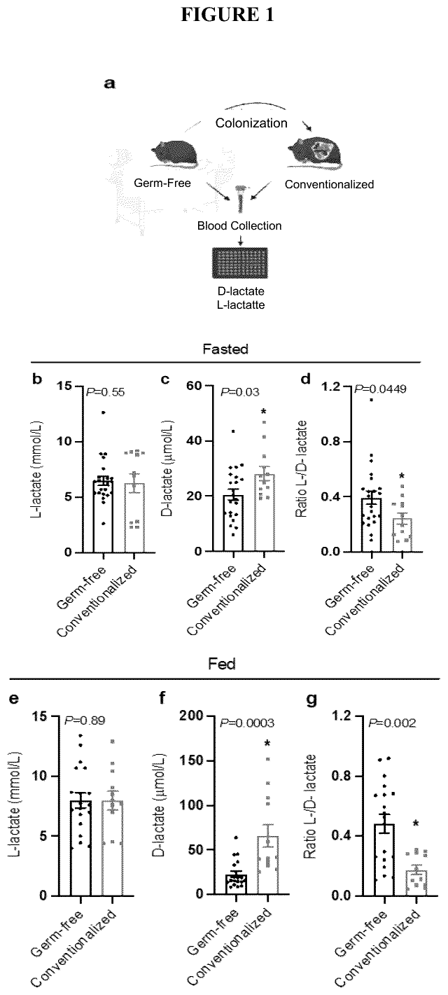

The gut microbiota is the main source of D-lactate, but not L-lactate, for the host. Germ-free and mice that were born germ-free, but then colonized (i.e., conventionalized) had comparable blood serum levels of L-lactate both during fasting and on a random-fed state (FIG. 1b,e). Conversely, significantly higher levels of blood serum D-lactate were found in conventionalized mice as compared to their germ-free counterparts in fasted and fed states, with a more pronounced difference noted in fed mice (FIGS. 1c, f). Accordingly, microbial exposure (i.e., conventionalization) significantly reduced the ratio of L-lactate/D-lactate in the blood serum in fasted and fed mice (FIGS. 1d, g). These data clearly demonstrate that gut bacteria are the main contributors to the circulating pool of D-lactate, but not L-lactate in the blood of mice. Our findings negate the concept of host-derived D-lactate via methylglyoxal detoxification as the main source of D-lactate in the body.

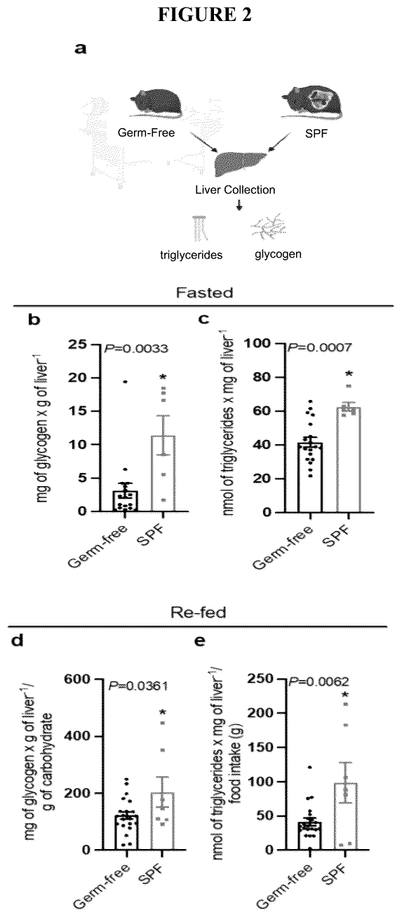

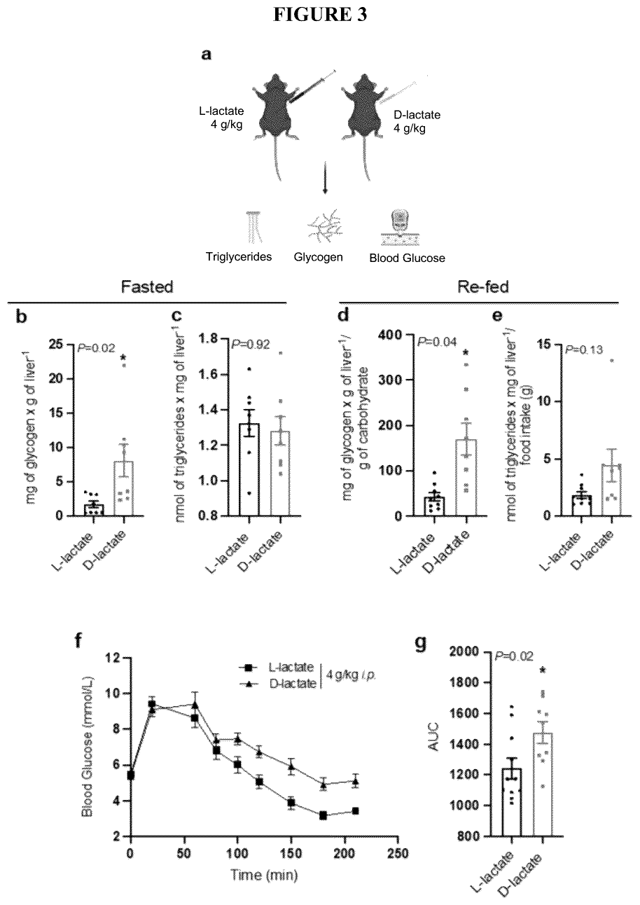

The gut microbiota regulates host liver glycogen and liver triglyceride content. Germ-free mice had lower liver glycogen and lower triglycerides in the fasted state compared to mice that were born germ-free, but then colonized (i.e., conventionalized) (FIGS. 2b, c). Germ-free mice had lower liver glycogen and lower triglycerides after re-feeding compared to mice that were born germ-free, but then colonized (i.e., conventionalized) (FIGS. 2d, e). Our findings show that gut bacteria contribute to the levels of liver carbohydrates (glycogen) and fat (triglycerides).

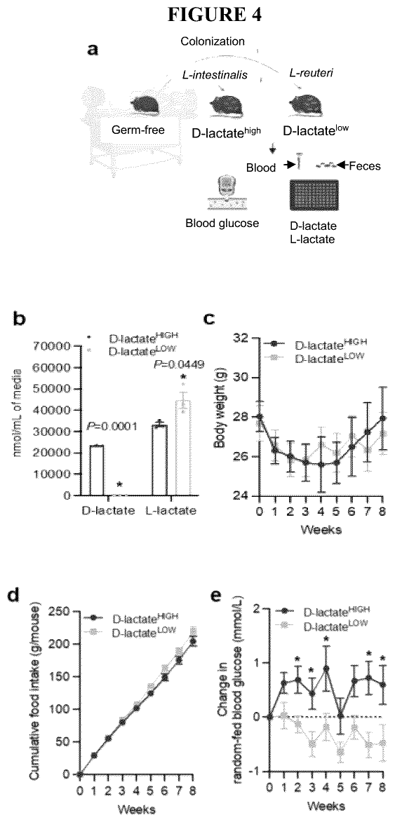

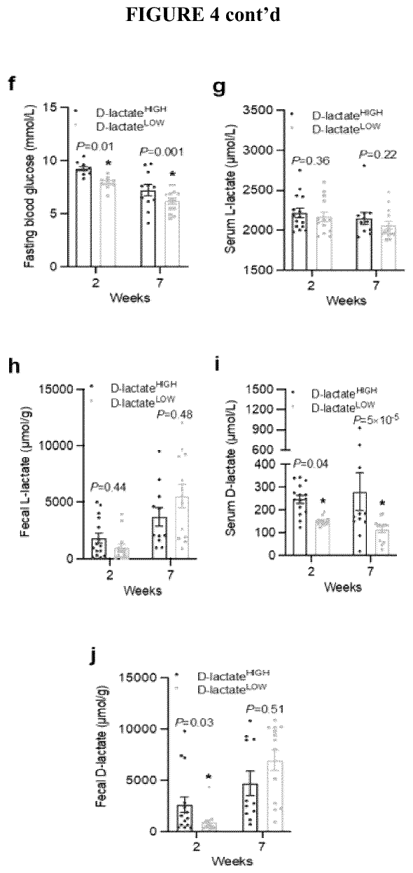

Microbial-derived D-lactate alters blood glucose in mice. We colonized mice with different strains of bacteria that produce high amounts of D-lactate or low amounts of D-lactate. We found that colonizing mice with a bacteria strain that produces a high amount of D-lactate increased serum and fecal D-lactate and increased fasting and random fed blood glucose (FIG. 4). These data show that microbial D-lactate is sufficient to alter blood glucose and that colonization of a mouse with a bacterial strain that produces higher D-lactate raises blood D-lactate and raises blood glucose.

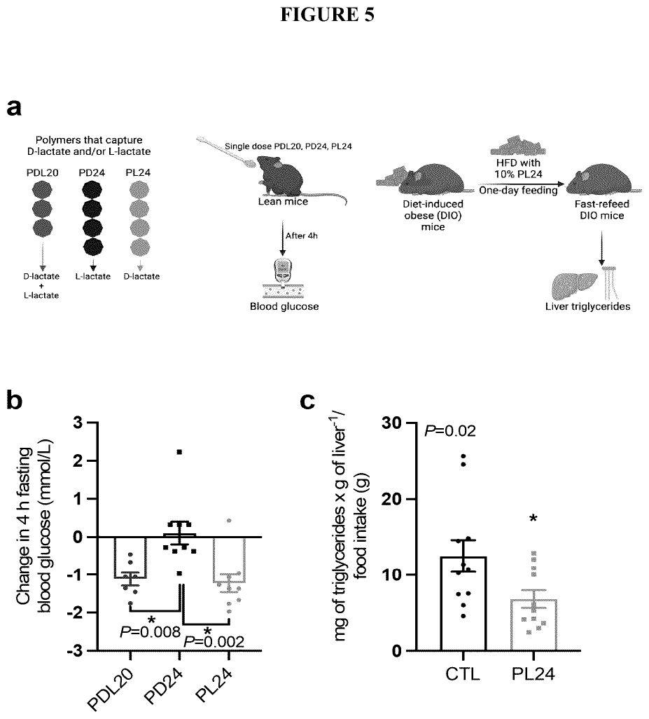

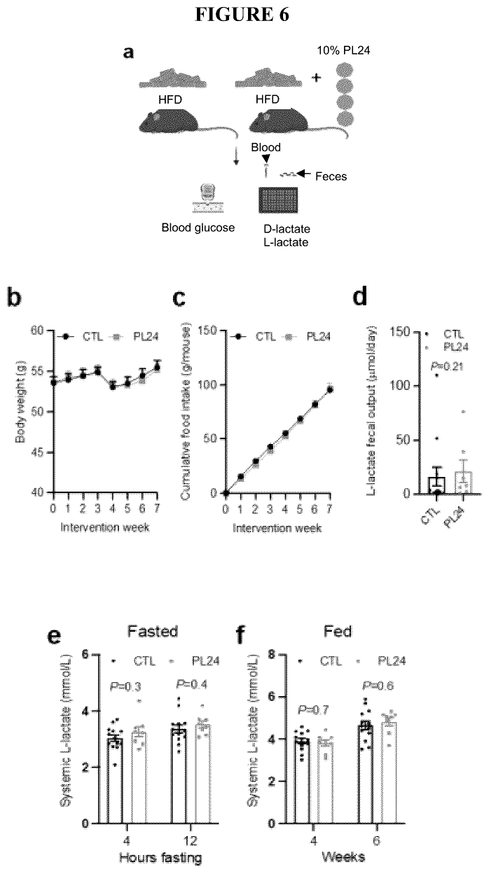

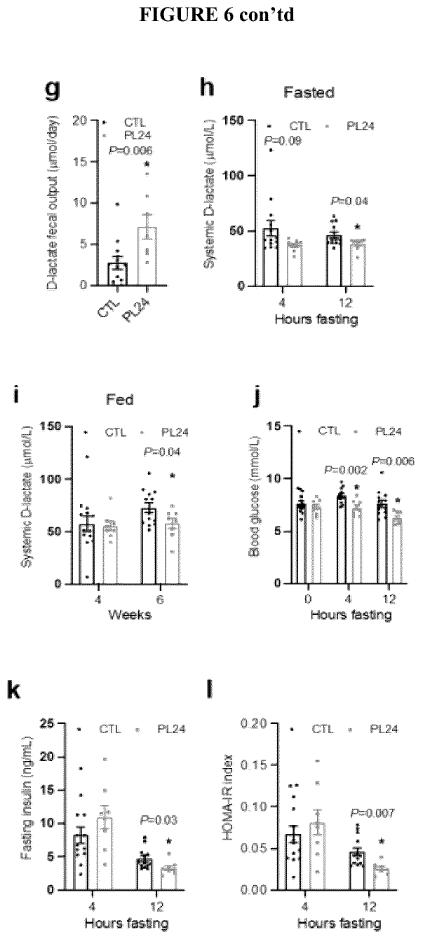

A gut microbial D-lactate trap lowers blood glucose and liver fat. We used biopolymers to capture D-lactate and/or L-lactate. We found that PDL20 (which captures D-lactate and L-lactate) and PL24 (which captures D-lactate) lowered blood glucose after oral delivery in mice (FIG. 5b). We also found that feeding PL24 lowered triglycerides in diet-induced obese mice (FIG. 5c). These results show that PL24 is a viable substrate trap that lowered blood glucose and liver fat in mice.

PL24 lowers blood glucose, insulin, and insulin resistance in diet-induced obese mice. Fasted mice were fed different biopolymers of lactate (i.e., polylactide) in order to trap gut microbial lactate in the intestine and prevent its absorption. It was hypothesized that this strategy would limit hepatic glucose production and the buildup of fat in the liver. This strategy is based on the fact that a polymer of L-lactate is a stereoisomer of D-lactate and therefore binds and sequesters D-lactate. Conversely, a polymer of D-lactate captures L-lactate, whereas a polymer of both D- and L-lactate sequesters both enantiomers of lactate (FIG. 2a). A significant reduction was observed in blood glucose 4 h after a single oral dose of poly-DL-lactide (PDL20) and poly-L-lactide (PL24), but not poly-D-lactide (PD24) to mice, suggesting that gut bacterial D-lactate is the main lactate enantiomer affecting host blood glucose (FIG. 2b).

The impact of long-term feeding with PL24 was next tested on the metabolic derangements associated with obesity. Diet-induced obese (DIO)-mice fed regular HFD and those fed HFD containing 10% PL24 showed no differences in body weight, fat mass and food intake throughout 7 weeks of dietary intervention (FIGS. 5b-c). Chronic feeding with PL24 did not affect the amount of L-lactate excreted in the feces and did not change blood levels of L-lactate in the fed or fasted state (FIGS. 5d-f). Conversely, mice fed HFD containing PL24 showed higher D-lactate fecal output (FIG. 5g) and lower D-lactate in the blood in the fed and fasted state (FIGS. 5h-i). Mice fed PL24 displayed lower fasting and random-fed blood glucose and insulin (FIGS. 5j-l), resulting in lower insulin resistance as indicated by the HOMA-IR index (FIG. 5l). These results show that PL24 is an efficient and specific gut substrate trap for D-lactate in mice, which lowers gut microbial D-lactate absorption and improves glucose and insulin homeostasis during obesity independent of changes in food intake or fat mass.

L-Lactate is almost always studied instead of D-lactate in mammalian metabolism. Mammals have (˜1000 times) higher levels of blood L-lactate. The Nobel prize winning discovery of the Cori cycle used “more efficient” D-lactate derived from bacteria obtained from a local collaborator. D-lactate appears long forgotten and there is considerable confusion on the importance and metabolism of D-lactate in mammals. The confusion over the importance of D-lactate in mammals has led to a dearth of knowledge of the metabolism of D-lactate and its role in metabolic disease. This is a key knowledge gap because people with diabetes and rodent models of diabetes both have higher levels of blood and urine D-lactate. These data reinforce the importance of D-lactate over L-lactate to the Cori cycle and that preventing the absorption of D-lactate, but not L-lactate, is sufficient to improve blood glucose regulation.

It is commonly assumed that the methylglyoxal pathway is the cause of higher D-lactate in diabetes, which results from increased flux through the methylglyoxal pathway. However, these data show that the main source of D-lactate is gut bacteria. This work adds a key component to the Cori cycle: gut microbial D-lactate. Importantly, this finding rendered possible to use a gut-trapping strategy to prevent D-lactate absorption and improve glucose homeostasis. This work establishes gut trapping of bacterial substrates as a promising strategy to treat obesity and metabolic diseases in the near future.

### Example 2

Twenty-three-week-old mice were on a 60% HFD for 17 weeks. After randomization, a quarter of mice were switched to 60% HFD+10% PL10, 60% HFD+10% PL38, and 60% HFD+10% PL65, respectively, the remaining mice were kept on 60% HFD for 7 weeks. Body weight (a) and cumulative food intake (b) were monitored weekly throughout the study. Serum L-lactate (c) and D-lactate (d) were measured at 4 h and 12 h of fasting. At week 9 of treatment, blood glucose (e) and insulin (f) were monitored during fasting, and insulin resistance was estimated by the HOMA-IR index (g). The number of independent biological replicates tested were: (a-e) CON n=10, PL10 n=9, PL38 n=10 and PL65 n=10; (f, g) CON n=9, PL10 n=9, PL38 n=10 and PL65 n=9. P values were calculated using Two-way repeated measures ANOVA with Bonferroni's multiple comparison test (a-g). Groups not sharing a common letter differ at P<0.05. The graphs depict the mean±SEM.

### Results

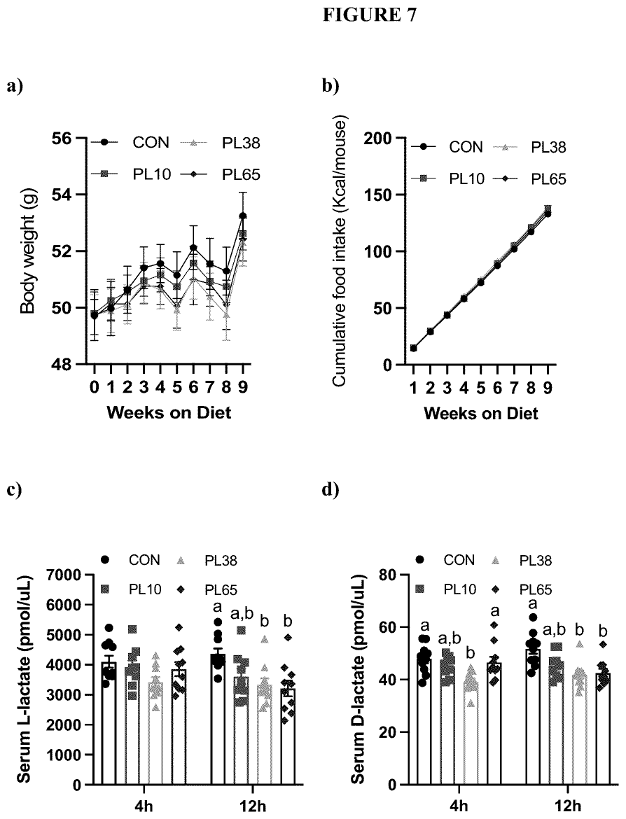

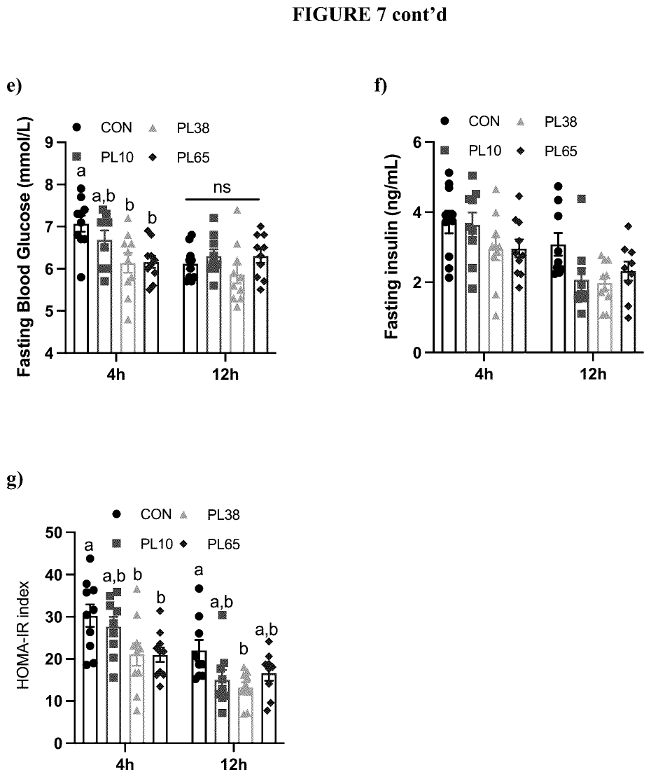

FIG. 7 shows poly-L-lactide lowers blood glucose, insulin and insulin resistance in a polymer length dependent manner in diet-induced obese mice. The optimized length of poly-L-lactide was tested on the same DIO mouse model. Mice fed regular HFD and HFD containing 10% PL10, PL38, or PL65 showed no differences in body weight and food intake throughout 7 weeks of dietary intervention (FIGS. 7a and b). Serum L-lactate levels were not affected by poly-L-lactide after 4 hours fasting but slightly decreased after 12 hours fasting with PL38 and PL65 (FIG. 7c). Serum D-lactate concentrations were significantly lowered by PL38 after both 4 and 12 hours fasting (FIG. 7d). PL65 decreased D-lactate level only after 12 hours fasting. PL10 has no effect on L- and D-lactate compared to control group. Mice that were fed PL38 and PL65 but not PL10 had lower 4 h fasting blood glucose (FIG. 7e) as well as HOMA-IR (FIG. 7g) despite no difference in fasting insulin (FIG. 7f). These results show that poly-L-lactide length does affect its ability in trapping D-lactate, leading to a poly-L-lactide length-dependent improvement in glucose and insulin resistance during obesity.

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR DOCUMENTS REFERRED TO IN THE APPLICATION

1. Tran, B. X., et al., Cost analyses of obesity in Canada: scope, quality, and implications. Cost Eff Resour Alloc, 2013. 11(1): p. 3.
2. Bilandzic, A. and L. Rosella, The cost of diabetes in Canada over 10 years: applying attributable health care costs to a diabetes incidence prediction model. Health Promot Chronic Dis Prev Can, 2017. 37(2): p. 49-53.
3. Mehran, A. E., et al., Hyperinsulinemia drives diet-induced obesity independently of brain insulin production. Cell Metab, 2012. 16(6): p. 723-37.
4. Lean, M. E. J., et al., Durability of a primary care-led weight-management intervention for remission of type 2 diabetes: 2-year results of the DiRECT open-label, cluster-randomised trial. Lancet Diabetes Endocrinol, 2019. 7(5): p. 344-355.
5. Madsen, L. R., et al., Effect of Roux-en-Y gastric bypass surgery on diabetes remission and complications in individuals with type 2 diabetes: a Danish population-based matched cohort study. Diabetologia, 2019. 62(4): p. 611-620.
6. Loomba, R., et al., Gut Microbiome-Based Metagenomic Signature for Non-invasive Detection of Advanced Fibrosis in Human Nonalcoholic Fatty Liver Disease. Cell Metab, 2019. 30(3): p. 607.
7. Pedersen, H. K., et al., Human gut microbes impact host serum metabolome and insulin sensitivity. Nature, 2016. 535(7612): p. 376-81.
8. Giles, D. A., et al., Thermoneutral housing exacerbates nonalcoholic fatty liver disease in mice and allows for sex-independent disease modeling. Nat Med, 2017. 23(7): p. 829-838.
9. Con, C. F. Cori., G. T., GLYCOGEN FORMATION IN THE LIVER FROM d-AND l-LACTIC ACID. J. Biol. Chem., 1929. 81: p. 389-403.
10. Con, C. F., MAMMALIAN CARBOHYDRATE METABOLISM. Physiol. Rev., 1931. 11: p. 143-275.
11. Thurston, J. H., R. E. Hauhart, and J. A. Schiro, Lactate reverses insulin-induced hypoglycemic stupor in suckling-weanling mice: biochemical correlates in blood, liver, and brain. J Cereb Blood Flow Metab, 1983. 3(4): p. 498-506.
12. Bueding, E. G., W., BLOOD CHANGES FOLLOWING GLUCOSE, LACTATE, AND PYRUVATE INJECTIONS INMAN. J. Biol. Chem., 1943. 147: p. 33-40.
13. Talasniemi, J. P., et al., Analytical investigation: assay of D-lactate in diabetic plasma and urine. Clin Biochem, 2008. 41(13): p. 1099-103.
14. Lin, M. H., et al., Determination of time-dependent accumulation of D-lactate in the streptozotocin-induced diabetic rat kidney by column-switching HPLC with fluorescence detection. J Chromatogr B Analyt Technol Biomed Life Sci, 2011. 879(29): p. 3214-9.
15. Jastroch, M., S. Ussar, and S. Keipert, Gut Microbes Controlling Blood Sugar: No Fire Required! Cell Metab, 2020. 31(3): p. 443-444.
16. Hasegawa, H., et al., Determination of serum D-lactic and L-lactic acids in normal subjects and diabetic patients by column-switching HPLC with pre-column fluorescence derivatization. Anal Bioanal Chem, 2003. 377(5): p. 886-91.
17. Goldberg, J. S., Stereocomplexes Formed From Select Oligomers of Polymer d-lactic Acid (PDLA) and l-lactate May Inhibit Growth of Cancer Cells and Help Diagnose Aggressive Cancers-Applications of the Warburg Effect. Perspect Medicin Chem, 2011. 5: p. 1-10.

1. A method of lowering at least one of blood glucose, liver fat, liver glycogen, insulin or insulin resistance in a mammal comprising administering to a mammal a polymer comprising L-lactate monomer, or a functionally equivalent derivative or fragment thereof, which inhibits or at least reduces D-lactate transport across the intestinal barrier.

2. The method of claim 1, wherein the polymer comprising L-lactate monomer is poly-DL-lactide and/or poly-L-lactide.

3. The method of claim 2, wherein the polymer comprising L-lactate monomer comprises between about 15-200 lactide monomer units.

4. The method of claim 2, wherein the polymer comprising L-lactate monomer comprises between about comprises 20-65 lactide monomer units.

5. The method of claim 1, wherein the polymer comprising L-lactate monomer is poly-L-lactide.

6. The method of claim 1, wherein the polymer comprising L-lactate monomer is linked or bound to non-biodegradable or non-digestible microparticles that promote dwell time in the intestinal lumen.

7. The method of claim 1, wherein a daily dosage the polymer comprising L-lactate monomer is administered in a daily dosage in a range of about 0.02 g/kg to 20 g/kg.

8. The method of claim 1, wherein the polymer comprising L-lactate monomer is administered together with one or more agents that promote degradation or metabolism of D-lactate.

9. The method of claim 8, wherein the agent that promotes degradation or metabolism of D-lactate is an enzyme selected from D-lactate dehydrogenase, D-lactate dehydrogenase (cytochrome), D-lactate dehydrogenase (cytochrome c-553) and D-lactate dehydratase (glyoxalase 3).

10. The method of claim 8, wherein the agent that promotes degradation or metabolism of D-lactate is a bacterium that expresses a D-lactate-metabolizing enzyme.

11. The method of claim 1, wherein the polymer comprising L-lactate monomer is administered together with a therapeutic agent useful to treat obesity, type 2 diabetes or fatty liver.

12. The method of claim 11, wherein the therapeutic agent is selected from the group consisting of a GLP-1-based drug, GLP-1/GIP co-agonists, GLP-1/GIP/glucagon tri-agonists, SGLT2 inhibitor, metformin and a statin.

13. The method of claim 1, wherein the polymer comprising L-lactate monomer is encapsulated within a hydrogel.

14. A method of lowering at least one of blood glucose, liver fat, liver glycogen, blood insulin and insulin resistance in a mammal comprising the step of inhibiting or at least reducing D-lactate transport across the intestinal barrier of the mammal by degradation or metabolism of D-lactate.

15. The method of claim 14, wherein the agent that promotes degradation or metabolism of D-lactate is an enzyme selected from D-lactate dehydrogenase, D-lactate dehydrogenase (cytochrome), D-lactate dehydrogenase (cytochrome c-553) and D-lactate dehydratase (glyoxalase 3).

16. The method of claim 15, wherein the agent that promotes degradation or metabolism of D-lactate is a bacterium that expresses a D-lactate-metabolizing enzyme.

17. The method of claim 14, wherein the mammal is obese, has non-alcoholic fatty liver disease (NAFLD), pre-diabetes or type 2 diabetes (T2D).

18. A composition comprising a polymer comprising L-lactate monomer in combination with one or more agents that promote degradation or metabolism of D-lactate.

19. The composition of claim 18, wherein the agent that promotes degradation or metabolism of D-lactate is an enzyme selected from D-lactate dehydrogenase, D-lactate dehydrogenase (cytochrome), D-lactate dehydrogenase (cytochrome c-553) and D-lactate dehydratase (glyoxalase 3).

20. The composition of claim 18, wherein the agent that promotes degradation or metabolism of D-lactate is a bacterium that expresses a D-lactate-metabolizing enzyme.

21. A composition comprising a polymer comprising L-lactate monomer linked or bound to non-biodegradable or non-digestible agent in combination with a pharmaceutically acceptable carrier.

22. The composition as defined in claim 21, wherein the non-biodegradable or non-digestible agent is selected from the group of beta-glucan soluble fiber, psyllium husk, cellulose, chitin, chitosan, guar gum, pectin, mucilage, locust bean gum, hydroxypropylmethylcellulose, arabinoxylan, alginate, inulin, inulin-type fructans, high amylose starch (resistant starch 2), galactooligosaccharide, polydextrose, resistant maltodextrin/dextrin, cross linked phosphorylated resistant starches 4 (RS4), glucomannan, acacia (gum arabic), plant cell wall fibers and polyethylene glycol.
